(19) United States
(12) Patent Application Publication
(10) Pub. No.: US 2005/0149268 A1
(43) Pub. Date: Jul. 7, 2005

(54) Methods of data analysis using ranks

(76) Inventor: Frank R. Sharp, Davis, CA (US)

Correspondence Address: DINSMORE & SHOHL, LLP, 1900 CHEMED CENTER, 255 EAST FIFTH STREET, CINCINNATI, OH, 45202, US

(21) Appl. No.: 10/496,915

(22) PCT Filed: Nov. 6, 2002
(86) PCT No.: PCT/US02/35540

Related U.S. Application Data
(60) Provisional application No. 60/334,014, filed on Nov. 28, 2001.

(51) Int. Cl. G01N 33/48; G06F 19/00; G06G 7/48

(52) U.S. Cl. 702/19; 702/20; 703/11

(57) ABSTRACT

Methods of analyzing data using ranks comprise ranking a set of captured data, comparing the ranked set of captured data to another set of ranked data, determining the change in rank between the sets, and defining the change in rank by statistical analysis.

### FIELD OF THE INVENTION

The present invention is directed toward methods of analyzing data using ranks. More particularly, the invention is directed towards a method of analyzing data comprising ranking a set of captured data, comparing the ranked set of captured data to another set of ranked data, determining the change in rank between the sets, and defining the change in the rank by statistical analysis.

### BACKGROUND OF THE INVENTION

With the sequencing of the human genome nearing completion, and with the advent of new microarray technologies, it is currently possible to assess large numbers of genes from tissue and blood samples (Brown & Botstein, 1999). It will soon be possible to assess the entire genome and eventually the entire proteome from blood and tissue samples of individuals with a wide array of medical, neurological and genetic disorders (Golub et al, 1999) (Staudt & Brown, 2000) (Schena et al, 1998). In addition, it should be possible to begin to define drug efficacy and drug side effects based upon these genomic and proteomic responses (Nuwaysir et al, 1999) (Puga et al, 2000).

There is a currently insurmountable problem when attempting to compare genomic and proteomic results between individuals, between different samples, and between different technologies for analyzing RNA or protein data. For example, just for the analysis of genomic responses using microarrays, it is currently impossible to compare results from microarrays that are fabricated using oligonucleotides (e.g., Affymetrix) compared to microarrays that are based upon cDNAs (e.g. Incyte) (Lockhart et al, 1996) (Khan et al, 1999) (Brown & Botstein, 1999) (Shalon et al, 1996). This problem is multiplied by the many different sets of technologies used to process and analyze this data, along with expression levels being measured differently depending upon the particular methods used (Kononen et al, 1998). Data may be consistent within a given microarray, but there is no current method for comparing data between different types of microarrays based upon different detection and measurement methods.

There is a substantial need for a convenient and accurate method to analyze data. There is also a need for a data analysis method which allows comparisons of data between different types of detection and measurement methods.

### SUMMARY OF THE INVENTION

Accordingly, it is an object of this invention to provide a convenient and accurate method to analyze data

In accordance with one aspect of the invention, there are provided methods of analyzing data using ranks. The methods comprise ranking a set of data captured, comparing the ranked set of captured data to another set of ranked data, determining the change in rank between the sets, and defining the change in the rank by statistical analysis.

The present methods are advantageous in providing a convenient and accurate method for analyzing data. Additional embodiments, objects and advantages of the invention will become more fully apparent in view of the following description.

### DETAILED DESCRIPTION

Though various procedures can adjust the average expression value on a chip or microarray to the same number, or can adjust values to a specific experiment or set of experiments, these methods are inadequate particularly for genes that are expressed at low levels, or genes that exhibit relatively small but extremely reliable changes in expression. The present inventor has found that ranking data based upon their expression value can be used to eliminate problems associated with comparing data between different detection and measurement methods. Methods in accordance with the present invention assign a rank to the data to be analyzed and ignore the absolute expression value for the remainder of the analysis.

More specifically, comparison between RNA chips, RNA microarrays, protein chips or RNA microarrays may be made. Comparisons of RNA array to protein array may also be made. The change in the rank of each gene or protein is compared and statistical analyses are performed to determine if there are significant changes in ranks in one condition, chip or set of chips compared to another. The only underlying assumption of this approach is that the identical set of genes/RNAs/or proteins is being assessed for all of the comparisons. The comparison can be performed for tens, hundreds, thousands or tens of thousands genes/RNAs/proteins as long as the same identical set is analyzed. This analysis does not require that the arrays contain the same genes/RNAs/proteins, only that the same set of genes/RNAs/proteins on different arrays are ranked and the comparisons made for the same sets of genes/RNAs/proteins.

The advantage of this method is that the absolute level of expression of a particular RNA or protein on any particular brand of chip, or any chip with any method of synthesis or analysis, can then be ignored. Instead, the rank of genes/RNAs or proteins on different expression platforms can be directly compared. This comparison is possible because the absolute level of expression is ignored, and hence all of the variability associated with extraction of the RNA or protein, the processing of the RNA or protein, or the measurement of the RNA or protein expression can therefore be ignored and can be discarded further analysis.

As an example of this technology, one can apply this approach to assessing changes in expression of the entire human genome or proteome. If it is assumed that humans have 70,000 genes, then the most highly expressed gene would be assigned a rank of 70,000. The lowest expressed gene would be assigned a rank of 1, with all other genes assigned a rank between 1 and 70,000 based upon their expression levels. The absolute expression of gene X on one chip might be 1000, while the expression of gene X on a separate chip might be 2000. However, with the present rank method, it is possible that gene X would be ranked 35,000 on both chips, and hence would be assigned a value of 35,000 on both chips based upon its rank with respect to the other genes on those chips. Therefore, expression values have been replaced with rank values that make the values assigned to gene X independent of its absolute expression value on each chip.

This now makes it possible to compare between any other chips, as long as the same identical set of genes/RNAs/proteins are being compared between the chips. Once genes/proteins are ranked on each chip/array to be compared, the rank values may then be compared to determine changes and used to perform a statistical analysis to define the changes. One such statistical analysis comprises a SAM analysis (Significance of Microarrays analysis), as described by Tusher et al, PNAS, Apr. 24, 2001, volume 98, pages 5116-5121, which is incorporated herein by reference. The SAM analysis is performed on the rank values rather than on the absolute expression values. The false discovery rate (FDR) can be set at 1, 5, or 10%, depending upon how stringent the data sets need to be. Once the most regulated genes, with the lowest standard deviations are described using SAM for a given disease, treatment or condition, then this set may be used to perform class prediction based upon a voting or other scheme as described by Golub et al, Molecular Classification of Cancer: Class Discovery and Class Prediction by Gene Expression Monitoring, Science 286: 531-537, which is incorporated herein by reference. This final analysis makes it possible to determine whether an unknown sample fits a particular disease gene profile or not with a pre-set level of confidence of, for example, 95%, 99%, or greater or lesser.

This genomic or proteomic analytical method makes it possible to analyze genomic or proteomic responses from blood or any other tissue (brain, skin, heart, lung, etc) using any type of microarray platform for oligonucleotides, cDNA, peptides, proteins, antibodies, or other detection approach. It also makes it possible to utilize data obtained using any detection method, including visible light, infrared, ultraviolet, fluorescence, or other method of detecting RNAs, cDNA, peptides, proteins, antibodies, or the like. The only constraint is that the identical set of RNAs, proteins, peptides, or small molecules are assessed in the comparisons. The analytical approach simply requires that the rank be used instead of the absolute expression value, and that the ranking be applied across identical sets of genes, RNAs, or proteins.

Additional embodiments and variations of the presently disclosed methods will be apparent to one of ordinary skill in the art in view of the present disclosure. The ranking approach may be used to compare the genomic responses in any tissue or organism for any medical, physiological, toxicological, or other treatment or condition. As long as the same (or related genes or proteins) are being examined, it may be possible to compare responses in yeast, bacteria, mammals and man using the ranking approach, even on the whole genomic or proteomic level. This may be done with assumption that many genes, RNAs and proteins would be excluded in man because they are not be present in lower species.

In addition, it may be possible to compare RNA and protein expression between individuals and between species using the ranking approach as long as the same RNA and protein species are compared. An example of such an analysis might show that ranks for RNA expression did not change for a specific treatment, like hypoxia. However, the ranks for a number of proteins might change dramatically with increases of ranks with hypoxia. This would provide evidence of no change in transcription, but a change in protein stability and increased protein expression as has been shown with hypoxia. This would provide a genomic and proteomic wide approach for looking for proteins that showing stabilization or destabilization following a specific treatment, drug or disease.

### EXAMPLES

The inventor has applied the ranking method to several different experimental analyses of microarray data.

### Example 1

The expression of over 13,000 genes is assessed on human Affymetrix microarrays for ten female patients and thirteen male patients. The lowest expressing gene is assigned a rank of I and the highest expressing gene a rank of 13,000, with all other genes on each microarray assigned a rank value based upon its expression value. A SAM analysis is performed to determine which genes have higher ranks in males compared to females, and which genes have consistently higher ranks in females compared to males.

Using this approach, 8 genes are identified with higher ranks in males compared to females, and 9 genes are identified with higher ranks in females compared to males. These genes are then subjected to a hierarchical cluster analysis and plotted for Females and Males. Of the genes that are highly ranked in the blood of males, 7 of 9 genes are known to be expressed on the Y chromosome. Using a conventional fold change approach, we were only able to detect 2 of these genes.

### Example 2

Children with new onset partial complex seizures have blood samples drawn prior to being started on any therapy. One month later, they have blood samples redrawn after having been placed on either valproate (VPA, also called Depakote) or on carbamazepine (CPZ, also called Tegretol). RNA expression in the blood samples of these patients is then analyzed on the human Affymetrix microarrays that assess 13,000 transcripts on each chip.

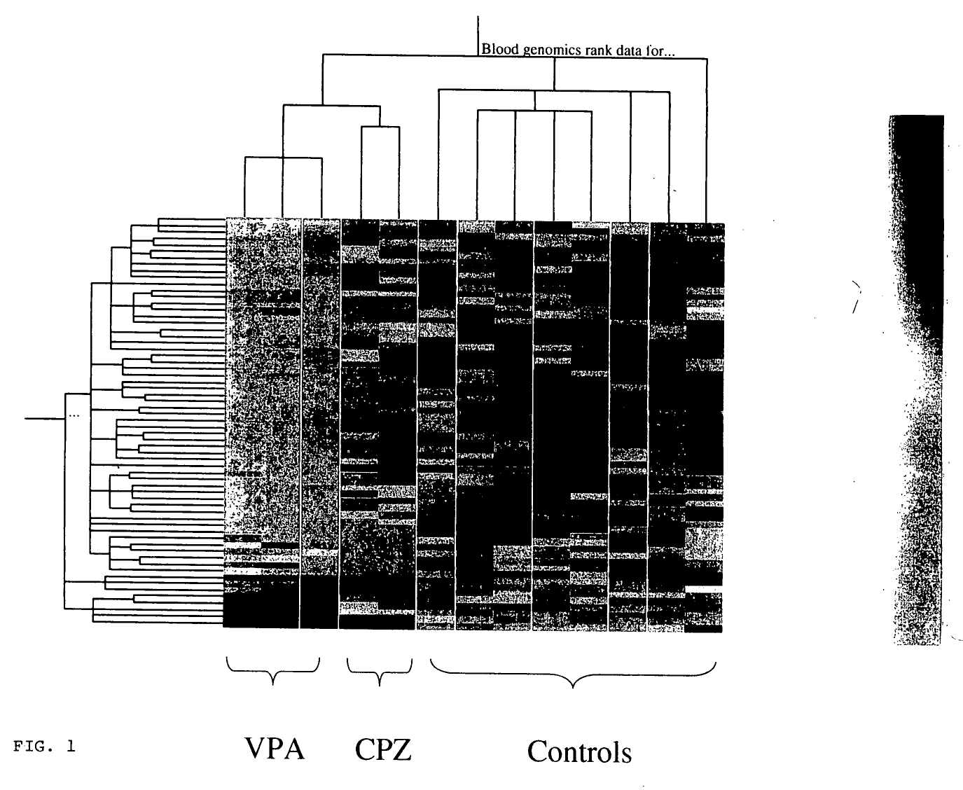

Gene expression levels on each chip are then converted to rank values and samples for VPA and CPZ compared to Controls. The genes that were most highly ranked and lowly ranked for VPA compared to controls were ascertained, and similarly the genes that were most highly ranked and lowly ranked for CPZ compared to controls were then derived. The cluster analysis of these genes is shown in FIG. 1. A specific pattern of gene expression for VPA patients is similar in all three patients, and is different from the two CPZ patients, and is different from the control blood samples.

### Example 3

The ranking approach is also used to assess blood samples from patients with Tourettes syndrome compared to control patients. Tourettes is usually an autosomal dominant disorder that begins in children and is associated with tics of the face and body associated with vocalizations. Blood is obtained from five Tourettes patients and 19 controls. Gene expression levels are obtained using microarrays as described. The gene expression levels are then ranked in accordance with the present methods.

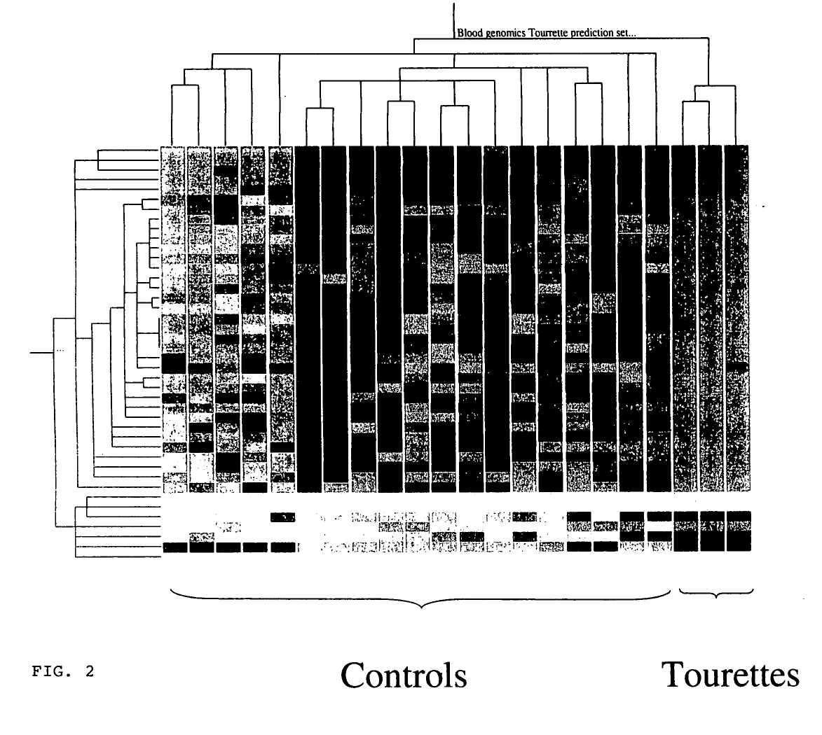

The genes that show lower ranks in Tourettes compared to controls were obtained and are shown in FIG. 2. For three of the Tourettes patients there was a specific pattern in which genes showing higher ranks and genes showing lower ranks compared to controls could be clustered into a specific pattern as shown on the attached plot for those three Tourettes patients.

The specific embodiments and examples set forth above are provided for illustrative purposes only and are not intended to limit the scope of the following claims. Additional embodiments of the invention and advantages provided thereby will be apparent to one of ordinary skill in the art and are within the scope of the claims.

1. A method of analyzing data comprising the steps of:
a. ranking a set of captured data;
b. comparing the ranked set of captured data to another set of ranked data;
C. determining the change in rank between the sets; and
d. defining the change in rank by statistical analysis.

2. A method according to claim 1, wherein the captured data is genomic, proteomic, or combinations thereof.

3. A method according to claim 1, wherein the captured data is obtained from blood or tissue.

4. A method according to claim 1, wherein the set of captured data comprises hundreds, thousands, or tens of thousands of genes, proteins, RNAs, small molecules, or mixtures thereof.

5. A method according to claim 1, wherein the captured data comprises data derived from a microarray platform for oligonucleotides, cDNA, peptides, proteins, antibodies or combinations thereof.

6. A method according to claim 1, wherein the captured data comprises data derived from employing visible light, infrared light, ultraviolet light, or fluorescence, for detecting RNAs, cDNA, peptides, proteins, antibodies or combinations thereof

7. A method according to claim 1, wherein ranking the set of data captured comprises assigning a rank to a gene, RNA, protein or small molecule according to a corresponding expression value.

8. A method according to claim 7, wherein the rank comprises an alphanumeric designation.

9. A method according to claim 1, wherein comparing the ranked data to another set of ranked data comprises comparing identical sets of genes, proteins, RNAs, small molecules, or combinations thereof.

10. A method according to claim 1, wherein comparing the ranked data to another set of ranked data comprises comparing RNA chips to microarrays, protein chips to microarrays, RNA arrays to proteins arrays, or combinations thereof.

11. A method according to claim 1, wherein defining the change in the rank by statistical analysis comprises determining if there are significant changes in ranks in one condition, chip or set of chips as compared to another condition, chip or set of chips, respectively, and determining if the data set fits a particular genomic or proteomic profile.
